(19) United States
(12) Patent Application Publication
(10) Pub. No.: US 2016/0367375 A1
(43) Pub. Date: Dec. 22, 2016

(54) Shoulder Replacement with Enhanced Glenoid Fixation

(71) Applicant: Craig BouIris, Alamo, CA (US)

(72) Inventor: Craig Boulris, Alamo, CA (US)

(21) Appl. No.: 15/143,997

(22) Filed: May 2, 2016

Related U.S. Application Data
(63) Continuation of application No. PCT/US2014/063681, filed on Nov. 3, 2014.

(51) Int. Cl. A61F 2/40; A61B 17/16

(52) U.S. Cl. CPC A61F 2/4081; A61B 17/1684; A61F 2220/0016

(57) ABSTRACT

Shoulder replacement: Enhanced fixation glenoid implants are disclosed. The glenoid implant designs presented herein have fixation projections that provide implants with better biomechanical properties than existing designs. Peripheral pegs have design features that provide better pull-out strength and stability. The central post can have stacked barbed discs, flutes, and surface features that provide better stability of the implant and can be self-centering. Better stability will lead to more reliable bony ingrowth and long-term survival of implants. Implants, pegs, and posts may be sized to patients and may not require intraoperative assembly. Implantation of devices requires minimal bone sacrifice and may be performed in a cemented or cementless, press-fit fashion.

### PRIORITY CLAIM

This Continuation application is filed under 35 U.S.C. 111a claiming priority to International Application No. PCT/US2014/063681, filed 3 Nov. 2014, which claims priority to U.S. Provisional Patent Application No. 61/899,711 filed Nov. 4, 2013, entitled “Shoulder Replacement: Enhanced Glenoid Fixation,” Craig Boulris, Inventor. These applications are incorporated herein fully by reference as if separately so incorporated.

### BACKGROUND

Disease of the shoulder joint can be a source of pain and disability and represent significant medical and rehabilitative challenges. Degenerative conditions, such as arthritis, can be progressive, fail to respond to conservative measures and ultimately require surgical correction. Shoulder arthroplasty or replacement may be necessary to restore function and relieve pain.

### SUMMARY

The embodiments presented herein represent examples of a total shoulder replacement glenoid implant system with enhanced fixation features. The novelty of the designs lies in the specific details of the fixation projections (posts and pegs) from the backside of the glenoid implant body.

Anatomic shoulder replacement glenoid implants can have polyethylene bodies with convex backsides and concave articular sides of slightly varying curvatures and sizes. The designs presented herein do not describe a specific curvature or size and can be applied to a variety of curvatures and sizes. Anatomic shoulder replacement glenoid implant face may vary in shape along a spectrum from symmetric ovoid to egg/pear shaped. The designs presented herein are not limited to a specific glenoid implant face shape and may be applied to the spectrum of shapes.

The embodiments of an enhanced ingrowth glenoid system presented herein represent novel designs, that permit use of either an all-polyethylene central post and peripheral peg fixation or a hybrid polyethylene-porous metal central post and peripheral peg fixation. One set of instrumentation may be used for insertion and/or extraction of both the all-polyethylene design or the hybrid polyethylene-porous, metal designs. Extraction entails minimal additional bone sacrifice.

Central post length may be scaled to glenoid implant and patient bone dimensions; downsized in smaller size implants-patients and upsized in larger implants-patients. Central posts may insert into a three tiered, tapered drill hole in the bony glenoid. This tiered shape minimizes volumetric bone extraction in the depths of the V-shaped glenoid. These features (tapering and sizing to patient size) minimize the likelihood of perforation of the backside cortex or catastrophic splitting of the bony glenoid. The central posts form-fit the drill hole and are thus self-centering.

Peripheral pegs may be scaled to glenoid implant and native glenoid dimensions for better patient size match and decreased likelihood of backside cortex perforation. Peripheral peg features can improve cement interdigitation, increase implant pull-out strength and improve stability. Peripheral peg features also can improve bony interdigitation and can increase implant pull-out strength when inserted in a cementless fashion into a hole of slightly smaller diameter.

Unlike some previous systems, the components presented herein can be non-modular and therefore require no time-consuming intra-operative assembly. Therefore, quality control is not needed performed on the operative back table. Preassembly helps avoid operator assembly error and implantation of a device with material junctional defects, which may not be detected at all or not detected until final component assembly. The components in this system may be assembled in the factory, where final quality control may be performed before distribution.

Embodiments of the enhanced fixation glenoid implant system presented herein give the surgeon highly flexible new tools in glenoid replacement. The systems are more patient specific, are bone preserving, and streamline implantation and extraction. Embodiments of the systems can provide better fixation, durability and survivorship. Cemented and cementless options are included.

### BRIEF DESCRIPTION OF FIGURES

This invention is being described based on specific embodiments thereof. Other features and aspects of the invention can be appreciated with reference to the figures, in which:

FIG. 1 depicts a lateral view of an embodiment 100 of a glenoid implant having an improved central post.

FIG. 2 depicts a bottom view of an embodiment 200 of a glenoid implant as depicted in FIG. 1.

FIG. 3 depicts a superior oblique view of an alternative embodiment 300 of a glenoid implant.

FIG. 4 depicts a lateral view of an embodiment 400 of a glenoid implant having a metal central post tip.

FIG. 5 depicts a bottom view of an embodiment 500 of a glenoid implant having a metal central post tip.

FIG. 6 depicts a superior oblique view of an embodiment 600 of a glenoid implant having a central post tip.

FIG. 7 depicts a lateral view of an embodiment 700 of a glenoid implant having a metal central post tip and metal peripheral pegs tips.

FIG. 8 depicts a bottom view of an embodiment 800 of a glenoid implant as shown in FIG. 7.

FIG. 9 depicts a superior oblique view of an embodiment 900 as shown in FIG. 8.

FIG. 10 depicts a cross-sectional lateral view of an embodiment 1000 of a glenoid implant having metal central post tip and metal peripheral peg tips showing the metal central cores.

FIG. 11 depicts a cross-sectional lateral view of an alternative embodiment 1100 of a glenoid implant to that shown in FIG. 10.

FIG. 12 depicts a cross-sectional lateral view of another alternative embodiment 1200 of a glenoid implant to those shown in FIGS. 10 and 11.

FIG. 13 depicts a cross-sectional lateral view of another alternative embodiment 1300 of a glenoid implant.

FIG. 14 depicts a cross-sectional lateral side view of a still further embodiment 1400 of a glenoid implant.

FIG. 15 depicts a top view 1500 of a drill useful for insertion of a glenoid implant.

FIG. 16 depicts a lateral view 1600 of a drill as shown in FIG. 15.

### DETAILED DESCRIPTION

### Definitions

As used herein, the term “a” means one or more.

The term “comprising” means “includes but is not limited to.

The term “consists,” and “consists of,” means “includes but is limited to.”

The term “glenoid” means the portion of a shoulder blade (scapula) having a socket for articulation with the ball of the humerus.

Terms used in the singular include the plural, and terms used in the plural include the singular.

### Descriptions of Elements of the Figures

The following list is of different elements and their names as used herein.
101 Main Body
102 Convex Back Side
103 Concave Articular Side
104 Superior Peg, All Polyethylene
105 Inferior Peg, All Polyethylene
106 A-G Stacked Barbed Discs
   106A Superior Peg, Stacked Barbed Discs
   106B Inferior Peg, Stacked Barbed Discs
   106C Proximal Central Post, Stacked Barbed Discs
   106D Distal Central Post, Stacked Barbed Discs
   106E Superior Peg Base, Stacked Barbed Discs
   106F Inferior Peg Base, Stacked Barbed Discs
   106G Proximal Central Post Base, Stacked Barbed Discs
107 Barb-Flute-Barb Central Post
108 Middle Central Post, Flutes
   108A Flute Slit

Glenoid Implant Replacement with Metal Tip
109 Central Post, 2-Tier Metal Tip
   109A Metal Peg Tip, Solid Central Core
   109B Metal Post Tip, Solid Central Core
   110A Superior Peg, Metal Tip
   110B Inferior Peg, Metal Tip
111A-H Metal Tip, Solid Core
   111A Peg Metal Tip Core with Proximal Screw Attachment
   111B Central Post Metal Tip Core with Proximal Screw Attachment
   111C Peg Metal Tip Core with Proximal Helical Blade Attachment
   111D Central Post Metal Tip Core with Proximal Helical Blade Attachment
   111E Peg Metal Tip Core with Proximal Barbed Attachment
   111F Central Post Metal Tip Core with Proximal Barbed Attachment
   111G Peg Metal Tip Core with Proximal Textured Surface Attachment
   111H Central Post Metal Tip Core with Proximal Textured Surface Attachment
112A-D Metal Tip, Porous Coating
   112A Peg Metal Tip, Porous Coating with Flat Interface
   112B Central Post Metal Tip, 2-Tier Porous Coating with Flat Interface
   112C Peg Metal Tip, Porous Coating with Beveled Interface
   112D Central Post Metal Tip, 2-Tier Porous Coating with Beveled Interface

3-Tier Drill
113A Cannulated Drill, T-Handle Shaft
113B Cannulated Drill Bit, Tip
114 Cannulated Drill Base
115 Superior Peg Drill Hole
116 Inferior Peg Drill Hole
117 T-Handle

3-Tier Drill Bit
118 3-Tier Drill Bit
   118A Bit, Proximal Tier with Cutting Groove
   118B Bit, Middle Tier with Cutting Groove
   118C Bit, Distal Tier with Cutting Groove
119 Drill Base with Elevated Section

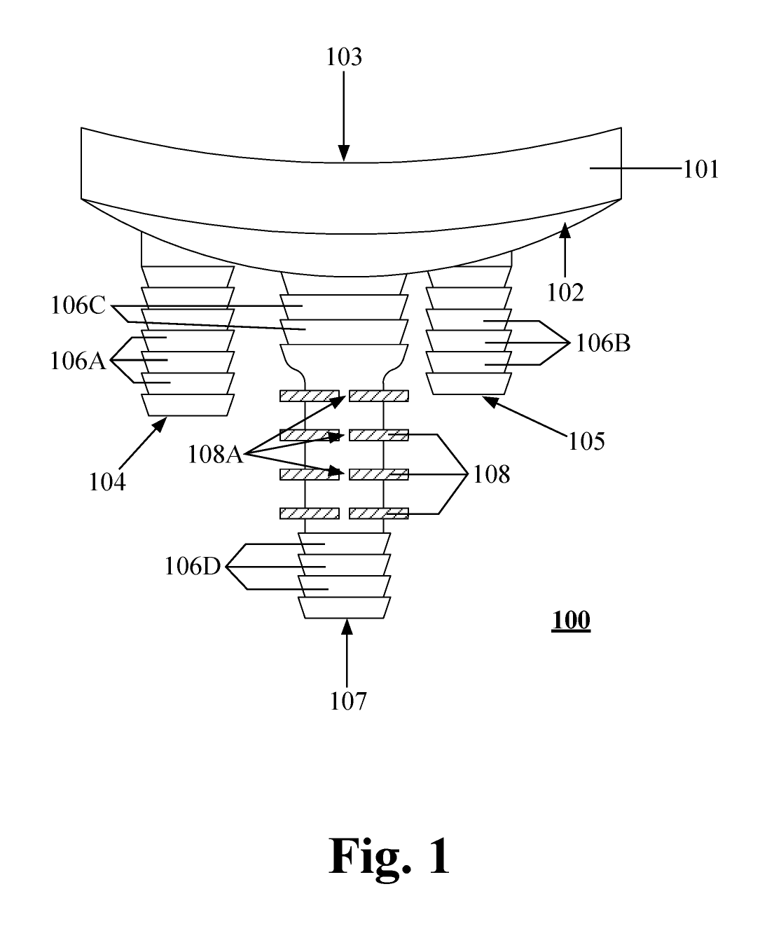

FIGS. 1-3 All Polyethylene Glenoid Implant: Barb-Flute-Barb Central Post

FIG. 1 Barb-Flute-Barb Central Post Design (Lateral)
101 Main Body
102 Convex Back Side
103 Concave Articular Side
104 Superior Peg, All Polyethylene
105 Inferior Peg, All Polyethylene
106A Superior Peg, Stacked Barbed Discs
106B Inferior Peg, Stacked Barbed Discs
106C Proximal Central Post, Stacked Barbed Discs
106D Distal Central Post, Stacked Barbed Discs
107 Barb-Flute-Barb Central Post
108 Middle Central Post, Flutes
108A Flute Slit

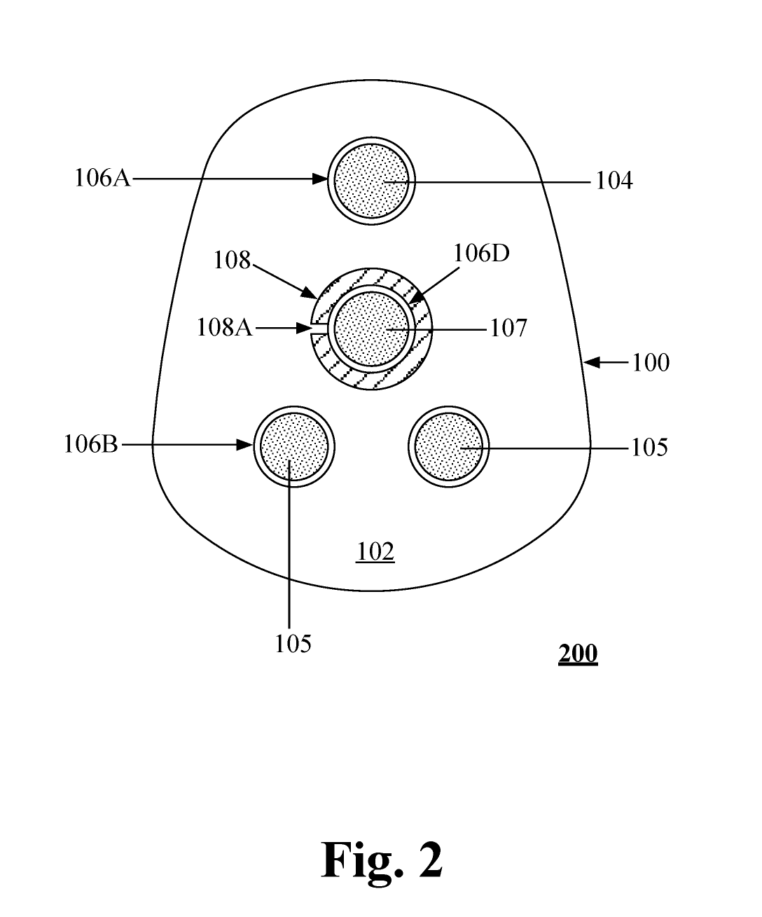

FIG. 2 Barb-Flute-Barb Central Post Design (Bottom)

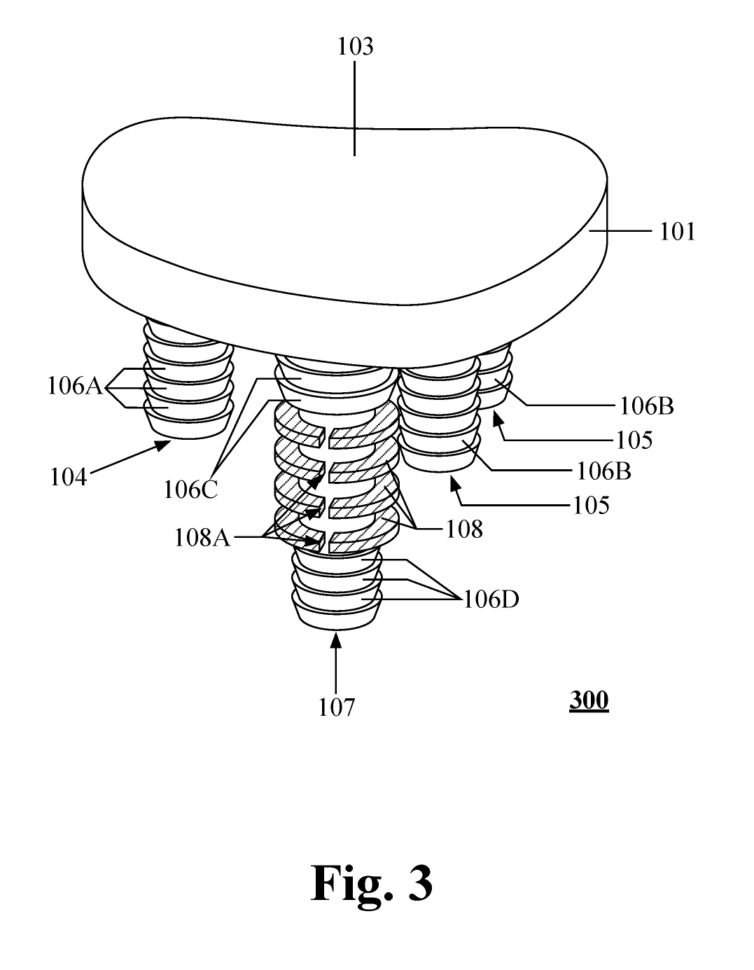

FIG. 3 Barb-Flute-Barb Central Post Design (Superior Oblique)

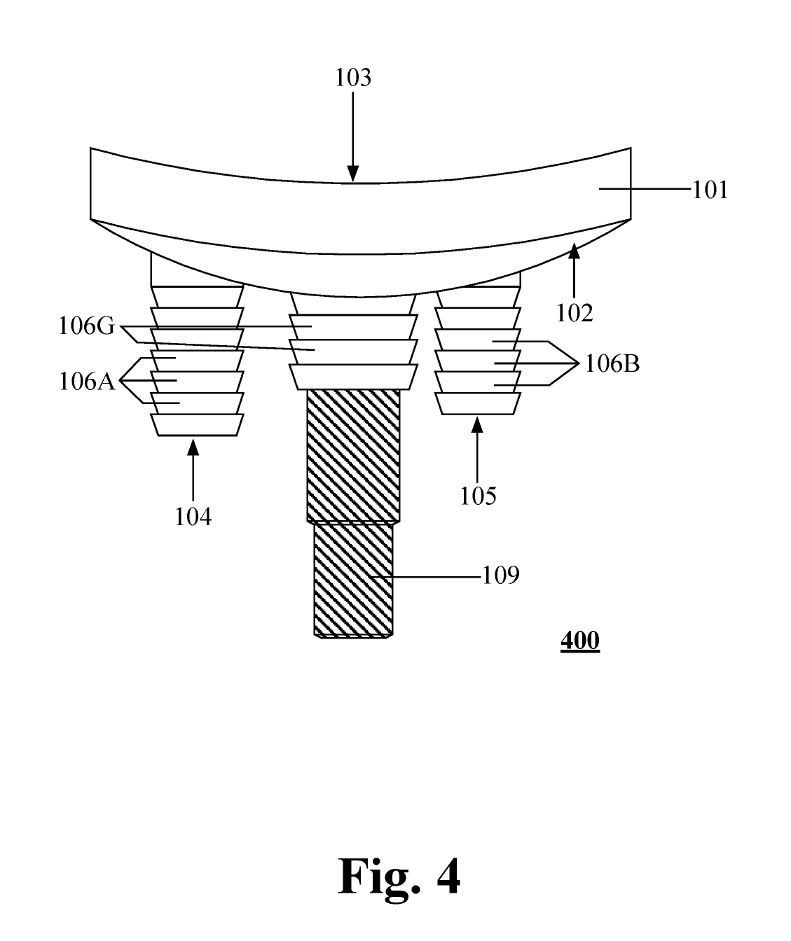

FIGS. 4-6 Hybrid Central Post: Polyethylene/Porous Metal Tip Central Post

FIG. 4 Hybrid Central Post Design (Lateral)
106G Proximal Central Post Base, Stacked Barbed Discs
109 Central Post, 2-Tier Metal Tip

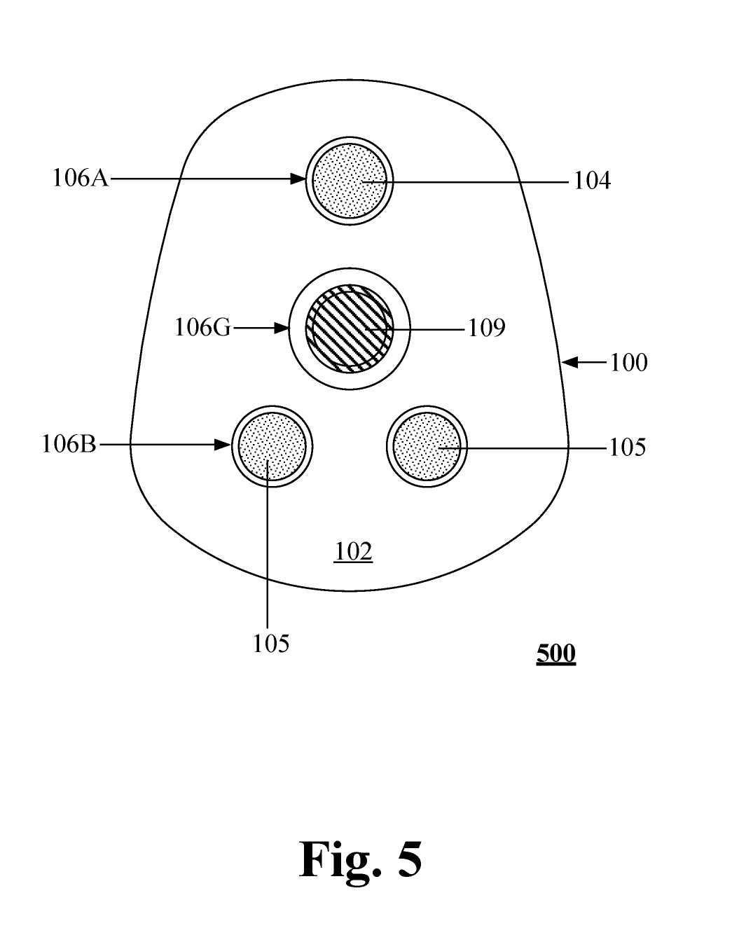

FIG. 5 Hybrid Central Post Design (Bottom)

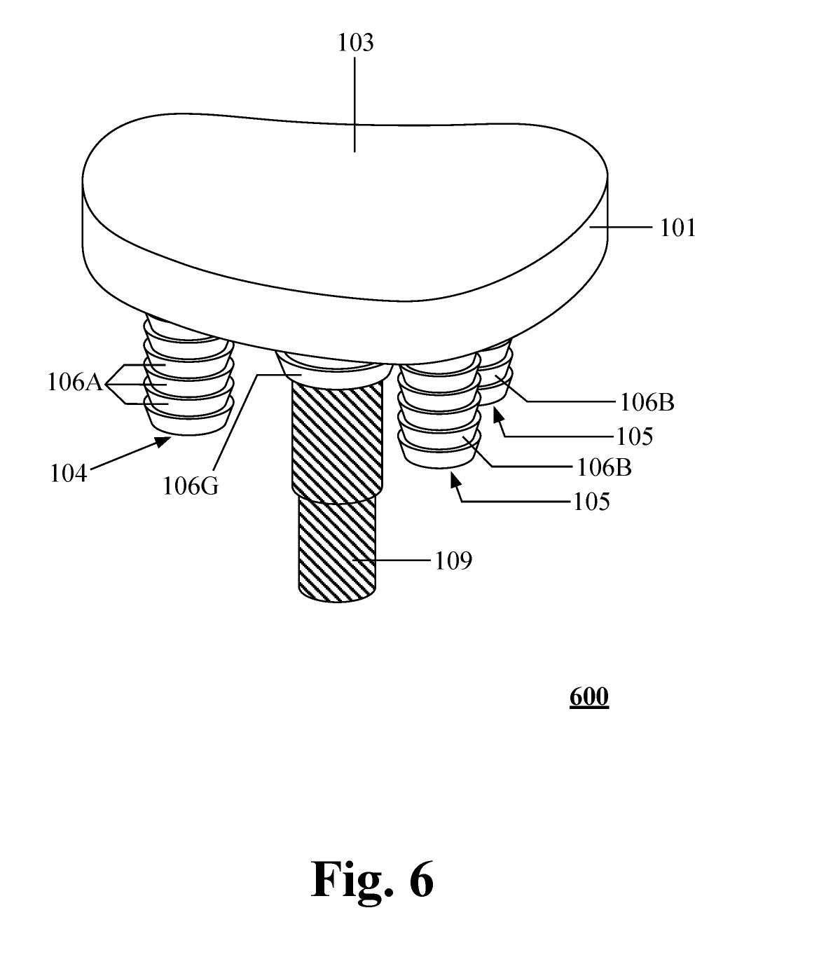

FIG. 6 Hybrid Central Post Design (Superior Oblique)

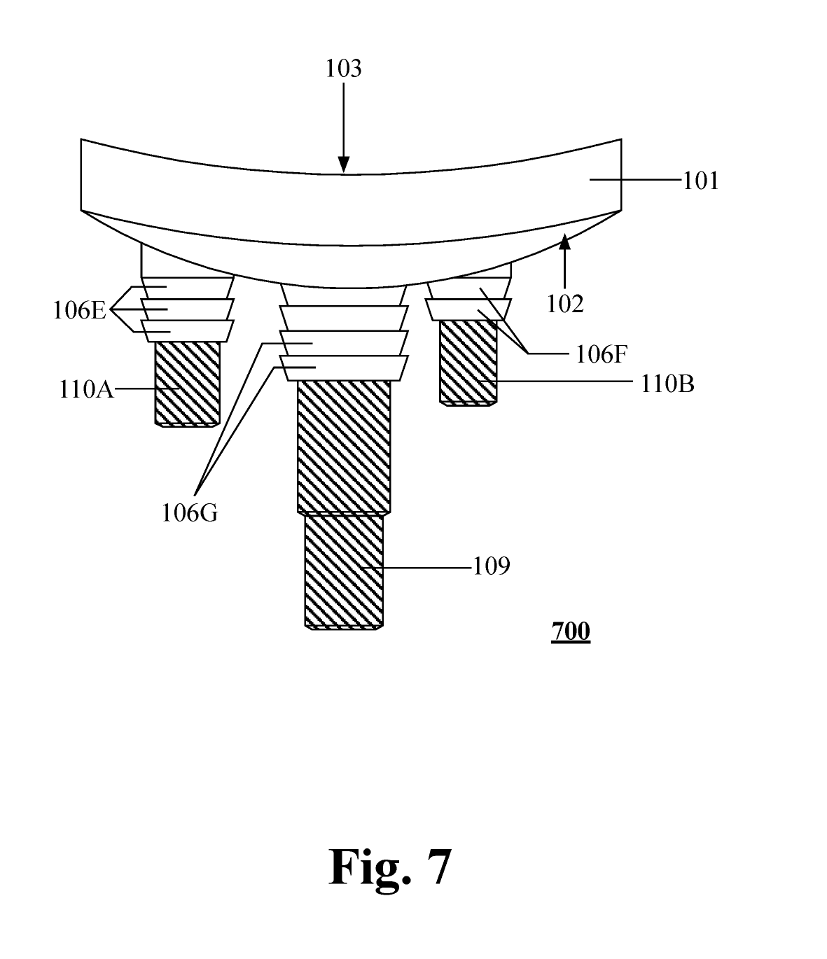

FIGS. 7-9 Total Hybrid Glenoid Implant: Polyethylene Base with Porous Metal Central Post & Porous Metal Peg Tips

FIG. 7 Hybrid Central Post & Hybrid Peg Design (Lateral View)
110A Superior Peg, Metal Tip
110B Inferior Peg, Metal Tip

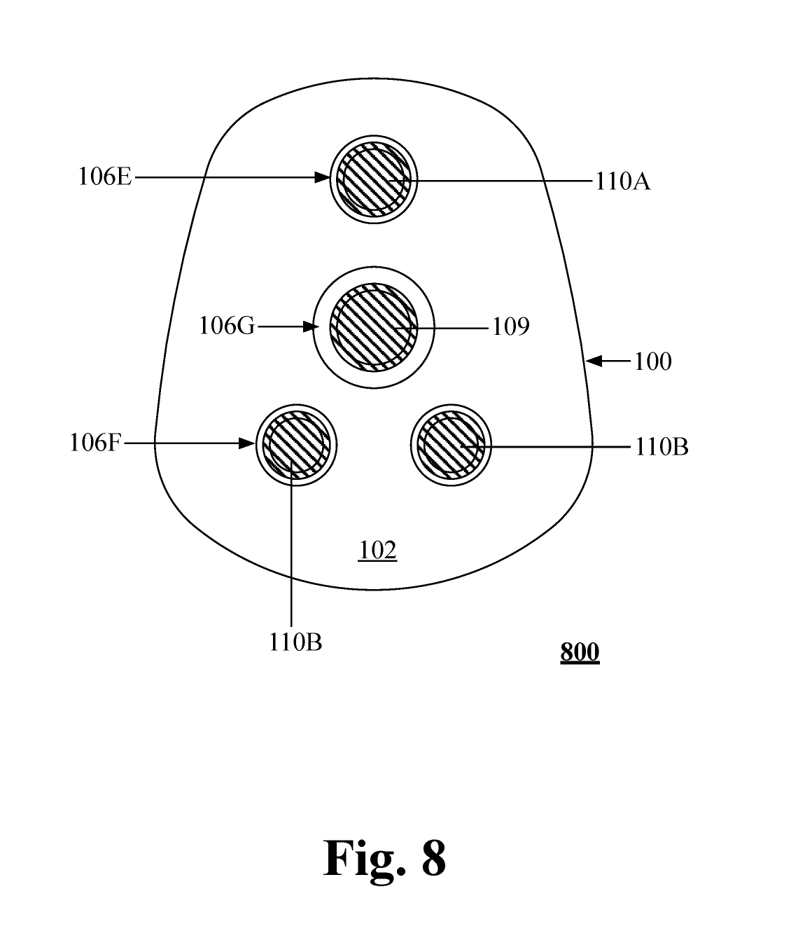

FIG. 8 Hybrid Central Post & Hybrid Peg Design (Bottom)

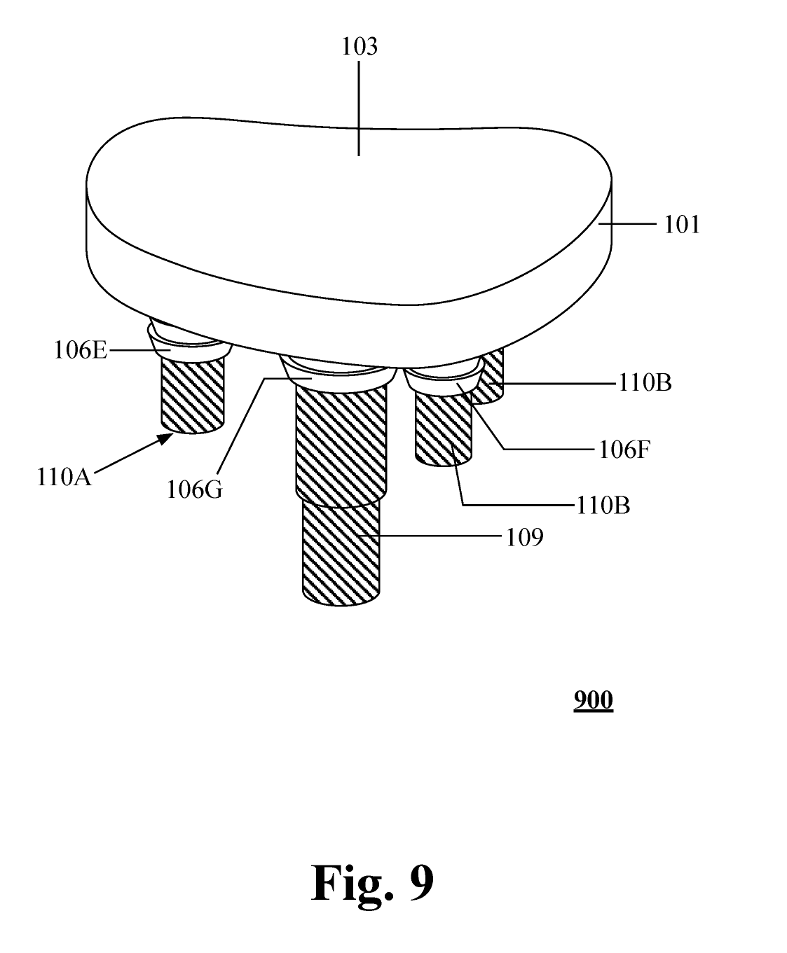

FIG. 9 Hybrid Central Post & Hybrid Peg Design (Superior Oblique)

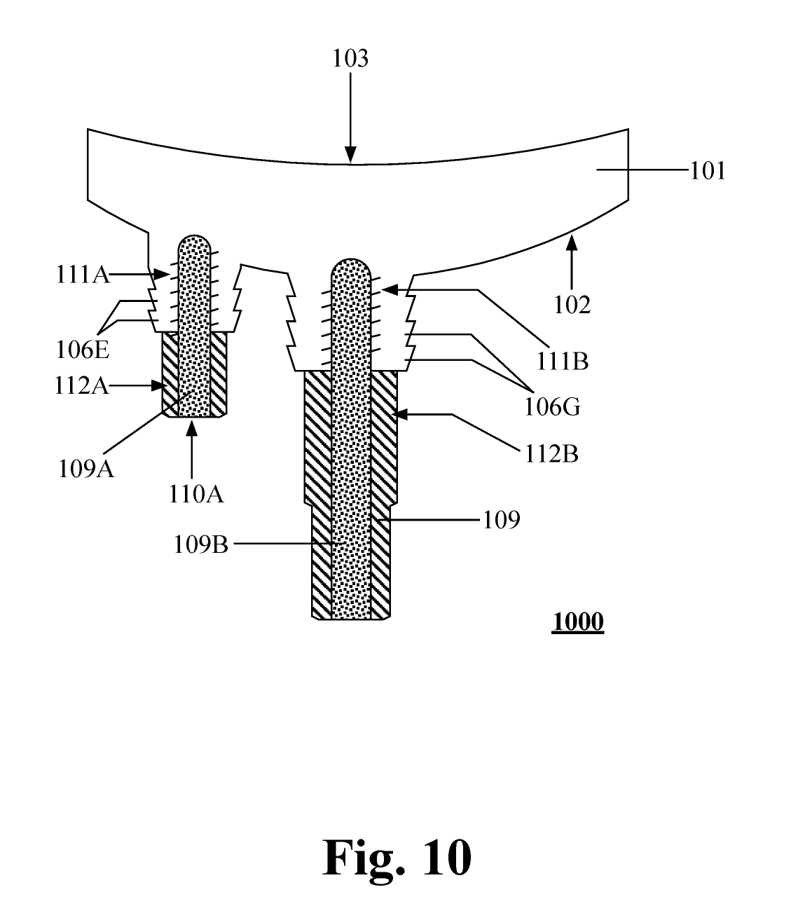

FIGS. 10-14 Hybrid Glenoid Central Post & Hybrid Peg Attachment (Lateral Cross Section)

FIG. 10 Metal Tip Threaded Screw Attachment, Flat Interface
109A Metal Peg Tip, Solid Central Core
109B Metal Post Tip, Solid Central Core
111A Peg Metal Tip Core with Proximal Threaded Screw Attachment
111B Central Post Metal Tip Core with Proximal Threaded Screw Attachment
112A Peg Metal Tip, Porous Coating with Flat Interface
112B Central Post Metal Tip, 2-Tier Porous Coating with Flat Interface

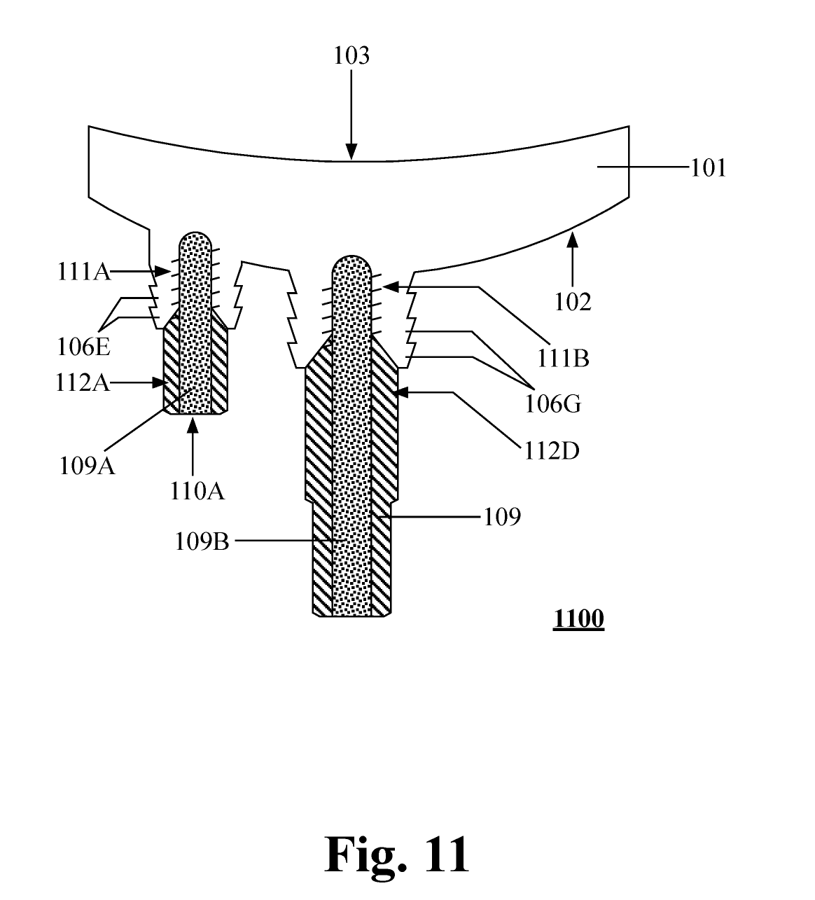

FIG. 11 Glenoid Implant with Beveled Interface
112C Peg Metal Tip, Porous Coating with Beveled Interface
112D Central Post Metal Tip, 2-Tier Porous Coating with Beveled Interface

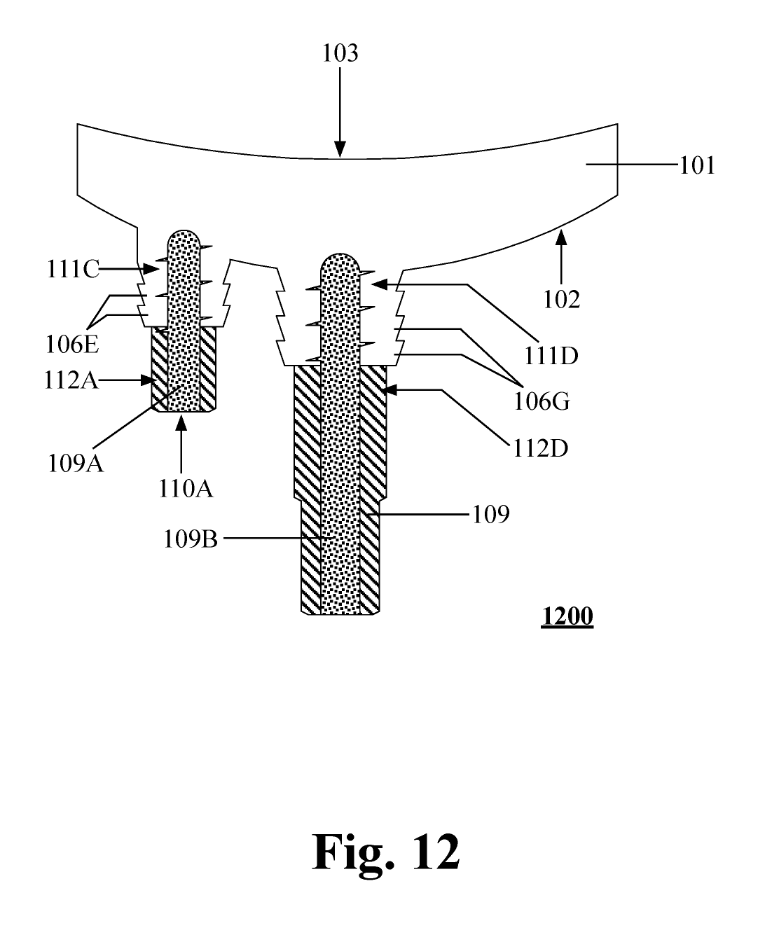

FIG. 12: Helical Blade Attachment
111C Peg Metal Tip Core with Proximal Helical Blade Attachment
111D Central Post Metal Tip Core with Proximal Helical Blade Attachment

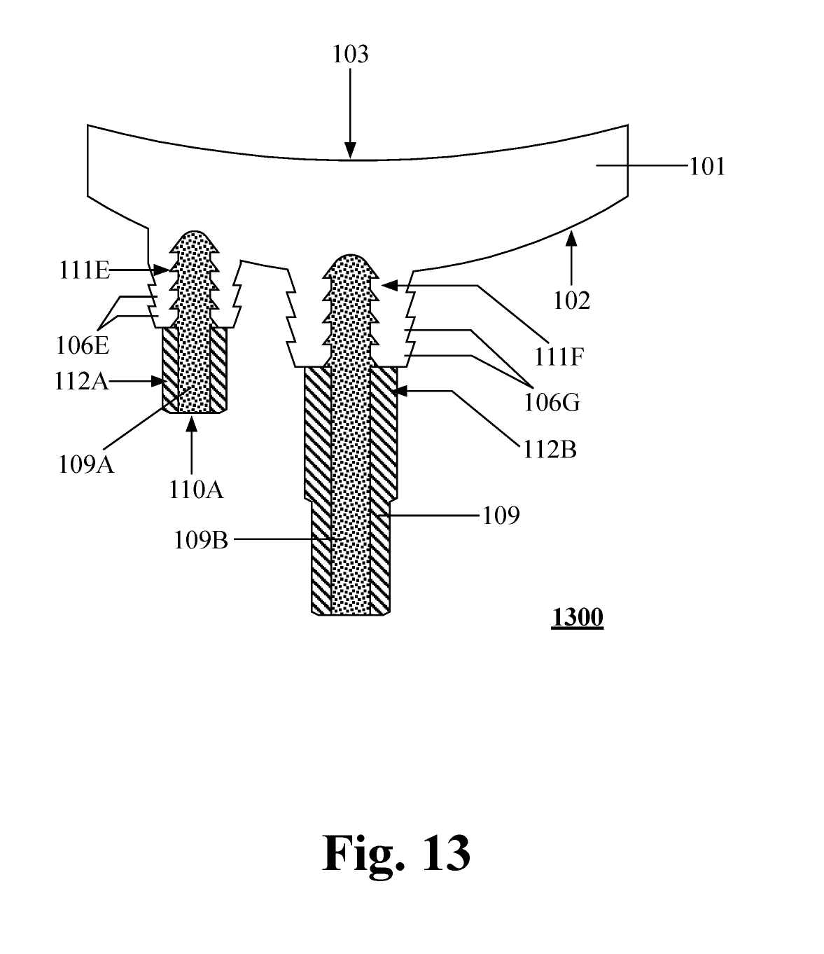

FIG. 13 Barbed Attachment
111E Peg Metal Tip Core with Proximal Barbed Attachment
111F Central Post Metal Tip Core with Proximal Barbed Attachment

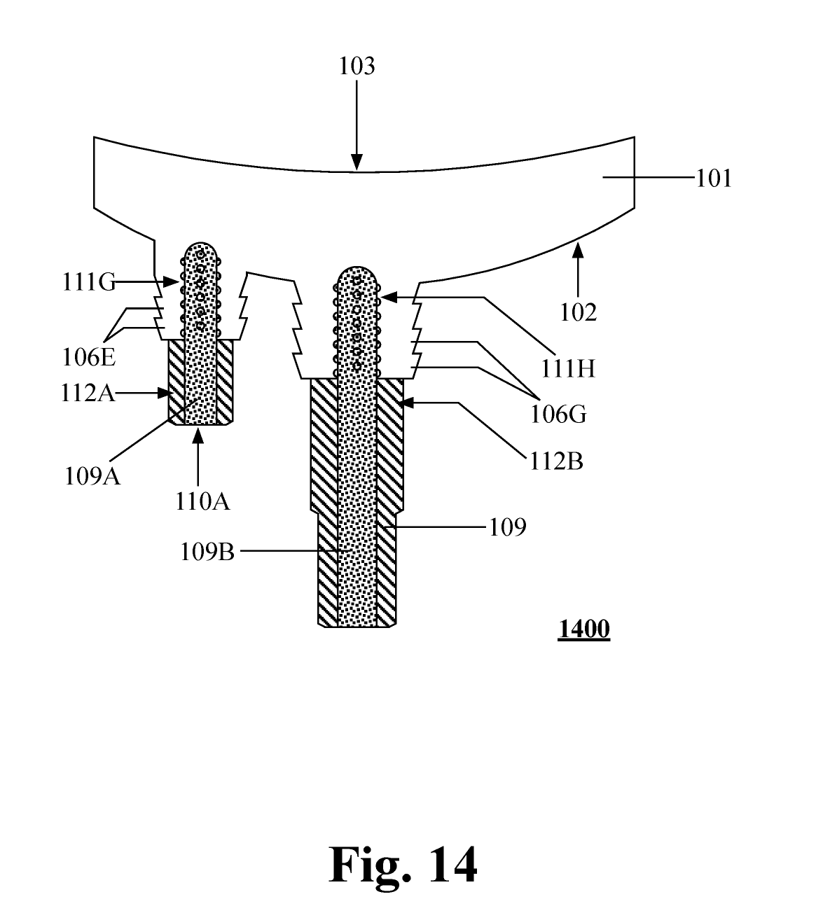

FIG. 14 Roughened Surface Attachment
111G Peg Metal Tip Core with Proximal Textured Surface Attachment
111H Central Post Metal Tip Core with Proximal Textured Surface Attachment

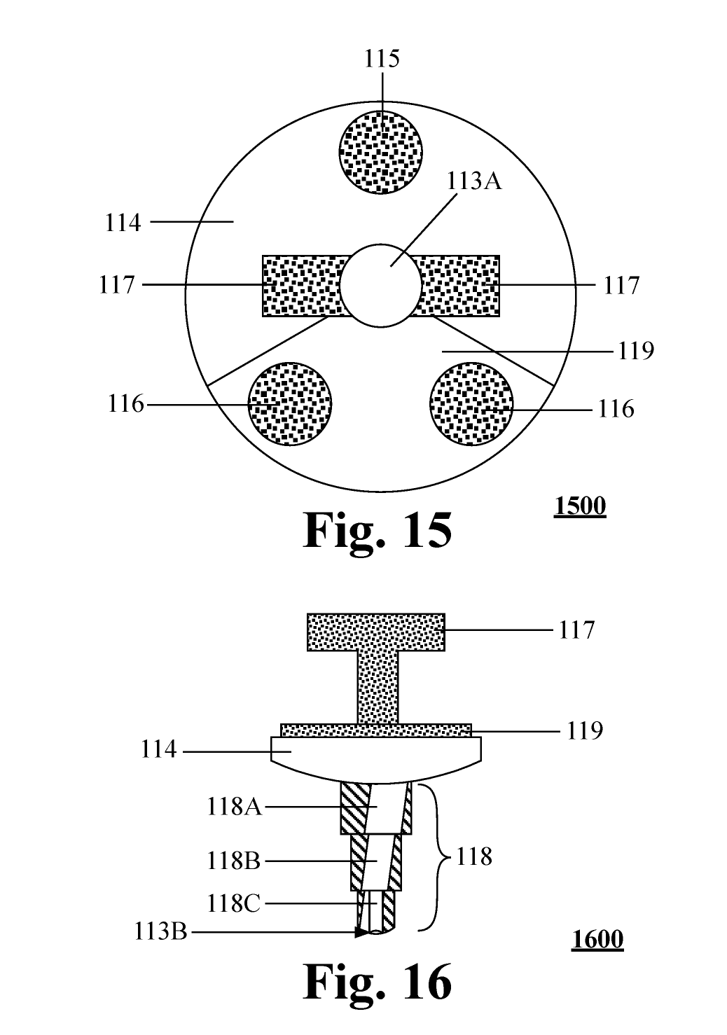

FIGS. 15-16 Common Cannulated Central Post Drill

FIG. 15 3-Tier Drill (Top view)
113A Cannulated T-Handle Shaft
114 Cannulated Drill, Base
115 Superior Peg Drill Hole
116 Inferior Peg Drill Hole
117 T-Handle
119 Elevated Section of Drill Base

FIG. 16 3-Tier Drill (Lateral View)
113B Cannulated Drill Bit Tip
118 3-Tier Drill Bit
118A Bit, Proximal Tier with Cutting Groove
118B Bit, Middle Tier with Cutting Groove
118C Bit, Distal Tier with Cutting Groove

### Aspects of the Invention

The humeral side (ball) of a shoulder replacement rarely loosens or fails, but should revision be necessary it can usually be done with relative ease. Revision of the socket side (glenoid) of the shoulder typically represents the greater challenge in shoulder arthroplasty. The glenoid implant may fail prematurely, causing pain. A scarcity of bone stock on the glenoid side of the shoulder joint often makes revision of the glenoid implant difficult. Maximizing fixation and durability of the initial glenoid implant is, therefore, of paramount importance. This invention represents an improvement in designs and components of glenoid implants with improved fixation and longer life.

Therefore, some aspects include a glenoid implant device, comprising:

a main body having a concave articular side and an opposite, convex side;

a central post affixed to said main body on said convex side, said central post having a cylindrical proximal portion having a first diameter, a middle cylindrical portion having a second diameter, and a cylindrical distal portion having a third diameter, said central post including:
a proximal portion having one or more coaxially arranged barbed disks thereon, at least one of said disks having a wider diameter proximally than distally on said central post;
a middle portion having one or more coaxially arranged flutes; and
a distal portion having one or more barbed disks thereon, at least one of said barbed disks having a wider diameter proximally than distally on said central post; and

one or more peripheral pegs, each of which is affixed to said main body on said convex side.

Additional aspects include a device of aspect 1, said first diameter being greater than said second diameter, and second diameter being greater than said third diameter.

Further aspects include a device of any of aspects 1 or 2, at least one of said peripheral pegs having one or more barbed disks thereon and having a wider diameter proximally than distally on said peripheral peg.

Yet further aspects include a device of any of aspects 1 to 3, at least one of said glenoid implant, said central post, said stacked barbed disks, said peripheral pegs, and said flutes comprising polyethylene.

Additional aspects include a device of any of aspects 1 to 4 where said glenoid implant is sized to fit the glenoid of a subject.

Still further aspects include a glenoid implant device, comprising:

a main body having a concave articular side an opposite, convex side;

a central post affixed to said main body on said convex side, said central post having a cylindrical proximal portion having a first diameter, a middle cylindrical portion having a second diameter, and a cylindrical distal portion having a third diameter, said central post including:
a proximal portion having one or more coaxially arranged barbed disks thereon, at least one of said disks having a wider diameter proximally than distally on said central post;
a middle portion comprising a metal and having a diameter smaller than the diameter of said proximal portion of said central post;
a distal portion comprising a metal and having a diameter smaller than the diameter of said middle portion of said central post; and

one or more peripheral pegs, each of which is affixed to said main body on said convex side.

Additional aspects include a device of aspect 6, at least one of said peripheral pegs comprising a metal.

Yet further aspects include a device of any of aspects 6 or 7, at least one of said peripheral pegs comprising a metal tip.

Still further aspects include a device of any of aspects 6 to 8, said metal portion of said central post and at least one of said peripheral pegs being affixed to a proximal portion by way of a metal core.

Additional aspects include a device of any of aspects 6 to 9, said metal core having a screw attachment to said central post or said peripheral peg.

Yet other aspects include a device of any of aspects 6 to 10, said metal core having a proximal helical blade attached to said central post or said peripheral peg.

Further additional aspects include a device of any of aspects 6 to 11, said metal core having a proximal barb attached to said central post or said peripheral peg.

Other aspects include a device of any of aspects 6 to 12, said metal core having a proximal textured surface attached to said central post or said peripheral peg.

Yet other aspects include a drill, comprising:

a handle;

a base having said handle attached thereto;

a 3-tiered cutting tip, having:
a proximal portion having a first diameter,
a middle portion having a second diameter; and
a distal portion having a third diameter;

said first diameter being greater than said second diameter; and

said second diameter being greater than said third diameter.

Further aspects include a drill of the prior aspect, said 3-tiered cutting tip further comprising a cutting groove.

### Glenoid Bodies and Central Posts

The glenoid implant designs presented herein can be composed of a polyethylene main body 101 as in FIGS. 1-14, with a concave articular side 103 as in FIGS. 1, 3, 4, 6, 7, 9, 10-14, and a convex backside 102 as in FIGS. 1, 2, 4, 5, 7, 8, 10-14. In the system presented herein, there may be a number of glenoid implant body sizes to match the spectrum of patient bony glenoid implant dimensions. Size of the central post may vary with size of the implant body. Use of a smaller body, with a shorter central post, in a small patient may prevent protrusion of the tip through the backside cortex and place the central post tip in a more optimal position for post osseointegration. Prevention of backside cortex protrusion may also help prevent catastrophic splitting of the glenoid. Use of a larger body, with longer post, in larger patient may provide a larger surface area for post osseointegration and place the central post tip deeper, yielding better stability and durability.

Fixation may be provided by an enhanced ingrowth Central Post 107, 109 as in FIGS. 1-14, one superior peripheral peg 104 and 106E/110A as in FIGS. 1-14 and two inferior Peripheral Pegs 105, and 106F/110B as in FIGS. 1-9. Central Post 107 may be either an all polyethylene fluted option FIGS. 1-3, or a hybrid consisting of a polyethylene base with a porous, metal tip 109 FIGS. 4-14. Peripheral Pegs 104, 105 may be either all polyethylene FIGS. 1-6, or a hybrid consisting of a polyethylene base with a porous, metal tip 106E/10A, 106F/10B FIGS. 7-14.

Fixation of a Central Post is enhanced by the fluted proximal portion or by use of a tiered Central Post being “self-centering” as described further herein.

### Peripheral Peg Design

Cemented Peripheral Pegs provide a major portion of the initial fixation and stability for glenoid implants with fluted or stacked barb Central Posts. Cemented peg pull-out studies show fixation varies considerably with small, subtle changes in peg surface features. Pull-out strength of pegs with a smooth surface is inferior to that of pegs with a dimpled surface, which is inferior to that of pegs with a slotted surface, which is inferior to that of pegs with a ridged surface. All existing finned/fluted glenoid implants employ peripheral peg surface features that could be improved upon.

In pull-out studies, when ridged pegs fail, the pegs fail at the cement-implant interface. The polyethylene ridge can deform away from the direction of pull-out, permitting peg displacement. Designs presented herein increase resistance to pull-out and resistance to deformation by shifting a polyethylene ridge mass away from the direction of pull out to form a barb. The resultant shape formed is no longer a symmetric V- or U-shaped ridge, but a beveled barb with its horizontal base proximally and angled surface distally. Barbs better resist deformation and pull-out than ridges. A barb may extend all the way around the circumference of the peg to form a barbed disc. Barbed discs in the designs presented herein can be stacked directly one on top of the other, without an intervening peg shaft, to provide a desired number of barbs per length of peg, thereby increasing pull-out strength. A stacked, barbed disc feature can be employed on the peripheral peg polyethylene surfaces in both the all-polyethylene and hybrid designs 106A, B, E, F presented herein as in FIGS. 1-14. Sizes of Peripheral Pegs may vary with size of the implant body, which itself may be size-matched to the spectrum of native glenoid morphology. Use of a shorter peg length in a small patient may place the barbs in a more optimal position for fixation and prevention of protrusion of the pegs through the backside cortex. Prevention of backside cortex protrusion helps to achieve better cement pressurization and helps avoid catastrophic splitting of the glenoid. Use of a longer peg length in larger patient may place the peg deeper for better stability and durability. Peg length may be scaled by either varying the number of stacked barbs, altering the size of each barbed disc or changing the length of the junctional region with the main body. Pegs may have one or more slots running down the length to facilitate more even cement pressurization between stacked barbs.

### Peripheral Peg: Cementless, Pressfit Application

A cement-free glenoid implant option can be desirable and provide a number of advantages. Cementless implantation avoids time-consuming cement mixture, application and curing. Cementless implants may avoid loosening problems associated as cement fatigue over time. Extraction and revision of a cementless device can be easier.

Inferior Peripheral Pegs 105, 106F/110B as shown in FIGS. 1-9 may be of shorter length than Superior Peg 104, 106E/110A as in FIGS. 1-14 to lessen risk of backside cortical perforation inferiorly, where glenoid bone depth under the peg tends to be shallower. Peripheral Pegs comprising of two or more polyethylene, stacked barbed discs can increase cementless pull-out strength and stability. Upon insertion into a bone hole, barbed edges of a peg and the surrounding bone can elastically deform slightly away from each other. Subsequent elastic recoil results in interdigitation of bone and barb. Pull-out is resisted at the interface between the barb base and bone, achieving improved stability. With successful press fitting of the Peripheral Pegs, the entire implant may be used in a cementless fashion.

A peg may have one or more slots running down the length of the polyethylene. In non-cemented implantation, a slot can improve bony interdigitation, providing rotational control.

### All-Polyethylene Glenoid Implant: Barb-Flute-Barb Central Post

Barb-Flute-Barb central post 107 has specific design features that improve central post cementless fixation FIGS. 1-3. Proximal Central Post 106C as in FIGS. 1, 2 and distal Central Post 106D as in FIGS. 1-3 may be composed of two or more stacked, barbed discs, similar in design to the peripheral pegs described herein. The proximal portion of a Central Post having stacked, barbed discs may be of larger diameter and height than the distal portion of Central Post stacked barbed discs. The larger junctional diameter proximally can result in both increased shear strength and resistance to bending compared to designs where proximal and distal post diameter is uniformly small.

The proximal portion of a Central Post and distal central post stacked barbed discs can have one or more slots running down the length of the polyethylene. A slot can improve bony interdigitation, providing increased resistance to rotational motion.

The intermediate or middle portion of a Central Post can be composed of two or more horizontal fins or flutes 108 as in FIGS. 1-3. Each flute may extend out horizontally from the middle portion of a Central Post, forming a flat disc of polyethylene. A space is created between adjacent flutes. Each flute may have one or more relaxing vertical slits 108A to prevent puckering or asymmetric deformation of the flute as it is inserted into a hole, creating more symmetric spacing between the flutes. Bone-forming cells (osteoblasts) can migrate into this space, or into the space above and below the flutes, where bone may form, thereby stabilizing and locking the Central Post inside the bony glenoid. Osseointegration is thereby achieved, with the increased stability and useful life of the glenoid implant.

A drill hole for the Central Post may have a plurality of tiers, for example, three steps or tiers; the smallest diameter being for the distal portion of Central Post stacked barbed discs 106D as shown in FIGS. 1-3, an intermediate diameter for the middle portion of Central Post flutes 108 as shown in FIGS. 1-3, and the largest diameter being for the proximal portion of a Central Post stacked barbed discs 106C as shown in FIGS. 1, 3. Drill holes may be slightly undersized relative to their respective implant counterpart, so that a tight form press-fit is achieved upon insertion. A rigid distal portion of a Central Post having stacked, barbed discs below the fluted area can stabilize and centralize the Central Post in the distal hole 106D as shown in FIGS. 1-3. Distal Central Post length may be slightly longer than the middle and proximal Central Post sections so that it is the first to engage upon insertion, centralizing the remainder of the Central Post. The proximal portion of a Central Post having stacked, barbed discs above the fluted area 106C as shown in FIGS. 1, 3 can provide stabilization and centralization of the Central Post in the hole proximally. The middle portion of flutes 108 of a Central Post, as shown in FIGS. 1-3 may be of larger diameter than the intermediate drill hole. With the post centralized above and below, the flexible flutes are centralized and may deform uniformly upon insertion into the middle hole.

In designs presented herein, both proximal and distal portions of a Central Post can comprise relatively rigid, stacked barbed discs to serve as a self-centering feature for the Central Post when inserted into the form fitting multi-tiered, or 3-tiered drill hole. This differs from prior fluted designs where there is no rigid component at the tip of the post. In those prior designs, flexible flutes that may deform non-uniformly are inserted down a drill hole of uniform diameter. There is no rigid component at the tip to form-fit with the distal hole, allowing the center of the distal tip to migrate. In those prior implants, the central post does not self-center. If bone density differs on one side the drill hole from the other, or if the implant is inserted at an angle, or if the large diameter distal drill hole perforates the side cortex, non-centering central post flute/fins designs may deviate to one side upon insertion. The flutes may deform asymmetrically, resulting in uneven horizontal spacing between flutes, leading to uneven osseointegration and compromised long-term stability. In contrast, with a centered Central Post, the implant can achieve better uniformity of spacing between flutes, more even osseointegration and better long-term implant stability and survivorship.

In systems presented herein, there may be a number of glenoid implant body sizes to match the spectrum of patient bony glenoid dimensions. Size of the central post may vary with size of the implant body. Use of a smaller body, with shorter central post, in a small patient may place the flutes in a more optimal position for osseointegration and prevent protrusion of the flutes through the backside cortex. Prevention of backside cortex protrusion may also help prevent catastrophic splitting of the glenoid. Use of a larger body, with longer post, in a large patient can provide greater surface area for interaction with bone and place the flutes and central post deeper for better stability, and durability. Adjusting post length may be achieved by either varying numbers of stacked barbed discs or flutes, altering the size of each stacked barb or changing the length of the junctional region with the main body.

Hybrid Central Post: Polyethylene Base with Metal Tip

Patient-specific variables, surgical training, and/or surgical preferences may make it desirable to implant a porous, metal central post, in preference to an all-polyethylene, fluted design. In certain embodiments, implants can allow for easy manufacture of a porous, metal Central Post. The main body, Peripheral Pegs and Central Post base may be similar to or identical to the all-polyethylene design 101, 102, 103, 104, 105, 106G as shown in FIGS. 4, 5, 6. A hybrid Central Post, however, can differ distally In place of the fluted middle region and distal stacked barbed region, these embodiments can have a 2-tiered porous metal Central Post. The 2-tiered tip 109 as shown in FIGS. 4-14 may be formed by application of a 2-tiered porous, metal coating 112B, 112D as shown in FIGS. 10-14 around a solid core of metal 109B as shown in FIGS. 10-14. A resultant 3-tiered hybrid design can allow for both reduced volumetric bony extraction in the depths of the central drill hole and ease of insertion in patients with tight exposure. The length of the distal metal tier may be slightly longer than the proximal metal tier and the proximal stacked barbed disc section allowing it to engage first upon insertion centralizing the remainder of the post.

A solid metal core 109B as shown in FIGS. 10-14 may extend up proximally into the polyetheylene Central Post base having stacked, barbed discs 106G as shown in FIGS. 4-14. A proximal portion of a solid metal core may have specific surface design features 111B,D,F,H as shown in FIGS. 10-14 that attach the metal tip to the polyethylene base. The features may include: screw threads 111B as shown in FIGS. 10, 11, multi-lead inclined threads forming a helical blade 111D as shown in FIG. 12, barbs 111F as shown in FIG. 13, raised textured surface 111H as shown in FIG. 14, or combinations of the above.

The junction of the proximal polyethylene Central Post and the porous metal coating 112B can be flat in nature, as shown in FIGS. 10, 12-14. An advantage of a flat junction is a longer length of proximal solid core for fixation to polyethylene. Junction 112D of the proximal polyethylene Central Post and the porous metal tip coating may be beveled in nature, as shown in FIG. 11. All solid core fixation options (including a threaded screw, a helical blade, barbed, or textured) can have a beveled interface. An advantage of a beveled interface is a more gradual transition zone with less concentration of stress at the junction joint and increased surface area for polyethylene/porous metal integration. Integration may be enhanced by heating the metal tip prior to and upon insertion.

There may be a number of glenoid implant body sizes to match the spectrum of patient bony glenoid dimensions. Size of the Central Post may vary with size of the implant body. Use of a smaller body, with shorter central post, in a small patient may place the central post tip in a more optimal position for osseointegration and prevent protrusion of the tip through the backside cortex. Prevention of backside cortex protrusion may also help prevent catastrophic splitting of the glenoid. Use of a larger body having a longer Central Post in a large patient can provide increased surface area for osseointegration and can place the Central Post tip deeper in the bone, thereby providing increased stability and durability. Central Post length may be adjusted for scale by varying either the dimensions of the polyethylene stacked barbed disc proximal base 106G as shown in FIGS. 4-14, or metal tip regions 109 as shown in FIGS. 4-14.

The metal tip's solid central core 109B as shown in FIGS. 10-14 may be used as a guide wire for extraction purposes. Unlike solid metal, porous metal can be morselized and extracted using a hardened drill. A cannulated, tiered central post drill as shown in FIGS. 15, 16 can be inserted over the central core, allowing removal of the porous coating and the entire tip with minimal volumetric extraction of additional bone.

A 3-tier drill as shown in FIGS. 15, 16 creates a drill hole of 3 different diameters. The intermediate diameter hole section and the smallest diameter hole section may accommodate a 2-tiered, porous, metal tip 109 as shown in FIGS. 4-14. The largest diameter section may accommodate the polyethylene proximal Central Post base having stacked barbed discs 106G as shown in FIGS. 4, 6, 7, 9, 10-14. A 3-tiered Central Post form can fit the 3-tiered drill hole and is self-centering.

The all-polyethylene and hybrid Central Posts may share common instrumentation and drills, allowing for seamless use of either all-polyethylene fluted or hybrid Central Post options.

### Total Hybrid Glenoid Implant: Porous Metal Central Post and Peripheral Pegs

Patient-specific variables, training and/or surgical preference may dictate implantation of an enhanced ingrowth glenoid implant where a Central Post and Peripheral Pegs can have metal tips. Designs presented herein can allow for easy manufacture of an all metal-tipped design option.

Metal tips 110A, 110B as shown in FIGS. 7-14 may be attached to one or more Peripheral Peg bases in similar fashions as the hybrid Central Post. A solid core of metal 109A as shown in FIGS. 10-14 may connect porous, metal tips to Superior Peg Base having stacked, barbed discs 106E as shown in FIGS. 7-14 and Inferior Peg Base having stacked barbed discs 106F as shown in FIGS. 7-9. A solid metal core extending up into the peg base may have surface design features that fixate and stabilize the attachment of the metal tip in the polyethylene base. Features can include screw threads 111A as shown in FIGS. 10, 11, multi-lead inclined threads forming a helical blade 111C as shown in FIG. 12, barbs 111E as shown in FIG. 13, a raised textured surface 111G as shown in FIG. 14 or combinations of the above.

A layer of porous metal, such as porous titanium may be applied over the solid central core. Porous metal may have a flat interface with the polyethylene base 112A as shown in FIGS. 10, 12-14 or a beveled interface with the polyethylene base 112 C as shown in FIG. 11. An advantage of a flat interface is a longer length of solid core for fixation to polyethylene. An advantage of the bevel is a more gradual transition zone with less concentration of stress at the junction joint and increased surface area for polyethylene/trabecular metal integration. Integration may be enhanced by heating the porous metal prior to and upon insertion.

Solid core 109A as shown in FIGS. 10-14 can be used as a guide wire for extraction purposes using a hardened cannulated drill, which morselizes porous metal.

Because of the porous nature of such a metal surface, Peripheral Pegs may be cemented into drill holes of a larger diameter with excellent fixation provided by the cement interdigitating in the metal pores. Porous metal-tipped Peripheral Pegs can also be used without cement and inserted scratch-fit into drill holes of a slightly smaller diameter. Peg pull-out is resisted at the porous metal-bone interface (as well as at the stacked barbed peg proximally), increasing stability. With a successful press-fit Peripheral Peg option, the entire implant can be cementless.

### 3-Tiered Cannulated Central Post Drill

Polyethylene (FIGS. 1-3) and hybrid (FIGS. 4-14) designs can employ common instrumentation and a common 3-tiered, Central Post drill 118 as shown in FIGS. 15, 16 for bony preparation or implant extraction. Each of the drill bit's 3 tiers can have one or more cutting grooves 118C distal tier with cutting groove, 118B, middle tier with cutting groove, 118A proximal tier with cutting groove. Each cutting groove may be aligned and feed into the groove above to help clear shavings and prevent clogging of a drill bit, and for extraction.

The drill 113A, 113B may be cannulated as shown in FIGS. 15, 16. Drill base 114 as shown in FIGS. 15, 16 can have a superior drill hole 115 shown in FIG. 15, and 2 inferior drill holes 116 as shown in FIG. 15 that serve as guides for a peripheral peg drill. The face can be stepped up 119 in the area of the two inferior peg holes for drilling for the slightly shorter inferior pegs as shown in FIG. 16. The proximal tier cutting groove(s) 118A can lie below one or more of the drill holes, 115, 116 to allow egress of bone shavings from the bit to prevent clogging. The drill can have a T handle 117 as shown in FIGS. 15, 16 to fine tune rotation for drilling the peripheral peg holes. The drill's cannulated T handle may insert over a standard driver, such as a hexagonal head screw driver, that can be magnetized if desired. A combined drill guide can expedite bony preparation of the glenoid, thereby allowing preparation of central and peripheral drill holes with one guide-bit. In certain embodiments, there can be a separate scaled tiered drill for each size implant post.

### Use of an Enhanced Glenoid Implant

During a total shoulder replacement procedure, the shoulder joint is exposed surgically and prepared. The humeral side preparation and replacement are performed according to routine methods and will not be further addressed here. The glenoid is exposed and prepared in the usual manner. A guide wire is inserted in a perpendicular fashion into the center of the bony glenoid socket and into the deep portion of the glenoid. The glenoid is sized to select the appropriate size for the glenoid implant. A cannulated reamer is used to ream the glenoid to the desired diameter and congruency.

A cannulated 3-tier drill as shown in FIGS. 15, 16, is then applied over the guide wire and used to ream a 3-tier hole in the bony glenoid for the Central Post. Cannulated drill T handle 117 as shown in FIGS. 15, 16 is then used to rotate the cannulated drill base 114 as shown in FIGS. 15, 16 and 3 peripheral pegs drill hole guides 115, 116 as shown in FIG. 15 into the desired position. Peripheral drill bits are placed sequentially into the 3 peripheral peg drill hole guides, creating holes in the glenoid to accept Peripheral Pegs. Peripheral Pegs may be either cemented or press-fit in the drill holes. If cementless Peripheral Peg fixation are chosen, a drill of slightly smaller diameter than the peg can be used to allow for press fit. If a cemented Peripheral Peg fixation is chosen, a drill of slightly larger diameter than the peg can be used to allow for a small cement mantle around the peg. A trial may be used to ensure adequate fit. In cemented fixation, cement is placed in the peripheral peg holes.

Either the all-polyethylene barb-flute-barb design as shown in FIGS. 1-3, hybrid Central Post design as shown in FIGS. 4-6, or total hybrid as shown in FIGS. 7-14 glenoid implants can be used. No intraoperative assembly need be required for implants. The central drill hole may not be filled with cement, as it accommodates the enhanced ingrowth Central Post. A chosen glenoid implant is then inserted fully, placing the Peripheral Pegs and Central Post into the appropriate drill holes, and impacted. If cement is used, the implant is held in position, waiting until the cement hardens and excess cement removed. If cementless application is chosen, the implant is inserted fully, aligning Peripheral Pegs and Central Post and holes, and impacted. Directly after full impaction, the surgeon may proceed without delay to the rest of the case. Humeral implant and shoulder closure are performed in the usual fashion.

### How an Enhanced Glenoid Works

Peripheral Pegs help provide initial fixation stability for the implant. Peripheral Pegs may be press-fit or cemented. In a cemented option, cement interdigitating between bone and the stacked barbed disc 106A, 106B, 106E, 106F as shown in FIGS. 1-14 or bone and the porous metal tip 110A, 110B as shown in FIGS. 7-14 hold Peripheral Pegs in position. In cementless options, press-fit between the bone and stacked barbed discs, or a scratch fit between the bone and porous metal can hold the peg in position.

A Central Post also provides initial bony fixation. In the fluted design, the proximal and distal stacked barbed regions 106C,106D as shown in FIGS. 1-14 press-fit into the proximal and distal drill holes. In the hybrid design, the stacked barbed discs proximally 106G as shown in FIGS. 4-14 press-fit in the proximal drill hole. 2-tiered porous metal tip 109 as shown in FIGS. 4-14 can be scratch fit into the middle and distal portions of a drill hole. A slightly longer distal section of a Central Post can engage the drill hole first, thereby centralizing the remainder of the post.

A Central Post can provide long-term fixation as bone grows into spaces on the Central Post, either between, above or below the flutes or into the porous spaces on the metal tip surface (osseointegration).

If total hybrid designs depicted in FIGS. 7-14 are used in a cementless fashion, the porous metal peg tips 110A, 110B as shown in FIGS. 7-14 also can provide long-term fixation through osseointegration. Together, the Peripheral Pegs and Central Post can fix the glenoid implant to the bony glenoid and account for improved implant survivorship.

### Manufacture of an Enhanced Fixation Glenoid

A single piece, all polyethylene fluted implant can be made by compression molding, ram-extruding, and/or otherwise engineering the main body, Peripheral Pegs and Central Post, as shown in FIGS. 1-3. In some embodiments, one can manufacture a glenoid implant using any other biocompatible polymer having sufficient strength, flexibility and wear properties. If a metal tip is to be added, the relevant polyethylene peg or post base 106E, 106F, 106G can be cut at the appropriate level as shown in FIGS. 7-14.

A metal tip can be constructed starting with a solid metal cylinder core 109A, 109B as shown in FIGS. 10-14 and containing a fixation feature proximally 111A-H (e.g., a screw, a helical blade, a barb, a textured surface, or combinations of these) as shown in FIGS. 10-14. Metal used in a central core can be titanium, stainless steel, cobalt chrome, tantalum, or other biocompatible metal. A metal tip outer layer can comprise any metal with an uneven surface into which bone can interdigitate. An outer layer can be made using titanium, porous stainless steel, porous cobalt chrome, porous tantalum, or other metal having suitable physical properties. This may be a porous, trabecular metal, a grit-blasted metal, spray coated (e.g., titanium), beaded (e.g., scintered or implanted), or otherwise textured metal. The interface area between the coating and the polyethylene base may be either flat 112A, 112B as shown in FIGS. 10, 11-14 or beveled 112C, 112D as shown in FIG. 11. Central Post metal tip 109B as shown in FIGS. 10-14 can have a 2-tiered porous coating applied over the solid core The Peripheral Peg metal tip can have a uniform diameter porous coating applied over the solid core 109A as shown in FIGS. 10-14. Polyethylene peg or post bases may have a pilot hole drilled in the center of the glenoid to accommodate a metal tip core attachment 111A-H as shown in FIGS. 10-12. If the threaded screw or helical blade fixation features are used, the pilot hole may be tapped. The metal tip may be heated above the melting temperature of polyethylene prior to and during the joining process to help the metal tip core cut through the polyethylene during insertion. With cooling, the polyethylene may re-harden around and into the core fixation features and porous metal, securely joining the metal tip and polyethylene base together.

A 3-tiered drill as shown in FIGS. 15, 16 can be made from carbon steel, titanium or other hardened metal.

### Advantages

Because of the finite lifespans of glenoid implants and the difficulty in removing or revising a glenoid implant, initial and long term fixation strength and stability, and therefore ultimate survivorship are important. Specific features of backside projections (posts and pegs) that determine initial and long term fixation, stability and survivorship of glenoid implants. Backside projections also determine an implant's ease of instrumentation, implantation or extraction and an implant's ability to match to a specific patient's size. Optimization of backside projection features enhance implant performance and desirability for a surgeon.

Prior, all-polyethylene glenoid implants relied entirely on cementation of backside projections for fixation and survivorship. Cement, however, eventually develops fatigue cracks. Cemented implants may loosen and require revision or removal. In order to minimize problems with cement fatigue and fixation failure, current generation glenoid designs employ a non-cemented central post. Central posts as described in embodiments were designed to allow bony ingrowth and osseointegration directly into spaces in the post. Both metal and polyethylene features can be incorporated into Central Posts for such purposes. Metal surfaces having porous cavities and polyethylene may have a series of flutes extending horizontally with intervening spaces. Bone can form between these voids, locking the post into the native bone. Depending on patient specific conditions (such as age, bone quality, bone quantity, osteoporosis) and surgeon preference, either metal or polyethylene features can be more desirable for a given patient and surgeon. Systems presented herein offer both porous metal and polyethylene fluteed options for enhanced, cementless Central Post fixation.

Failure of full osseointegration of prior fluted central post designs has been observed in a concerning number of study patients. Excessive micromotion can prevent bony healing and osseointegration in a variety of biologic situations involving bone-forming cells (osteoblasts) and can be a cause of failed osseointegration as has been seen in fluted designs. Micromotion can be more problematic in patients with asymmetric glenoid wear, where the implant may have to be seated at a nonanatomic angle of version. Larger shear forces across the implant cause this increased micromotion. Excessive micromotion and failure of bony osseointegration may compromise long-term survival of an implant. Insufficient initial central post and implant fixation can permit excessive implant micromotion and can be a cause of incomplete osseointegration. As a result, the use of previous fluted central post glenoid designs in patients with certain asymmetric wear patterns may be limited Improved central post fixation and stability can be desirable to improve implant osseointegration and survival of the implant, to raise confidence in the use of fluted designs, and to expand the surgical indications of fluted designs.

In prior fluted designs, flexible flutes serve two purposes. The first purpose is to act as the main stabilizer of the central post. The second purpose is to act as the feature that allows osseointegration of the central post. The prior, non-rigid, flexible flutes, however, are suboptimal for the purpose of stabilizing the post. More rigid features, including stacked barbed discs, and a multi-tiered Central Post as described herein can better stabilize the post. Moreover, in prior designs, a large percentage of total post length above and/or below the fluted region serves as a mere spacer, functioning neither in stabilization nor osseointegration.

In designs presented herein, every portion of the central post can be put to work either for stabilization or osseointegration purposes. Middle, fluted regions can enhance osseointegeration. Proximal and distal regions can function as stabilizers and centralizers.

Prior fluted central post designs may fail due to asymmetrical insertion into a bony drill hole. Prior flutes have larger diameters than the drill holes and thus require deformation in order for the implant to be inserted. This process goes well if the surrounding bone is of uniform density, adequate depth and the device is inserted in a perfectly straight trajectory. However, if bone is osteoporotic on one side of the hole, or extra dense on another side of the hole, the post may deviate to the weak side. A V-shaped glenoid is narrower at the deep base than superficially. Many glenoids have substantial superficial wear and may have less depth than the implant central post length. If a pilot drill perforates the outer cortex on one side of the drill hole, the flutes and post may deviate to the side of the perforation upon insertion. This deviation results in an off-centering of the post and creates a deforming rotational force. The deforming force may either prevent full symmetric seating of the implant body or act as a destabilizing force on a fully seated implant. The likelihood of such a deviation is increased the larger the distal hole diameter becomes, and the more distal the flutes are positioned on a post.

In designs exemplified and presented herein, flutes can be positioned proximally up the post to the middle section. The distal post and hole into which it inserts are of lesser diameter than the flutes. Both of these features decrease the likelihood of backside cortex perforation. A rigid, smaller diameter, region at the post tip, press-fit into the bottom of a tiered drill hole, also acts to center the post and implant. The flexible central flutes follow in line, avoiding potential asymmetric seating. Additionally, one or more slits in one or more flutes can permit the flute(s) to flex without buckling. Such flexing allows the central post to remain in a desired position within the drill hole, and to avoid asymmetric seating.

Peripheral Pegs of designs presented herein can be put to work to stabilize the implant. Stacked barbed discs can extend the entire length of a peripheral peg, and can provide improved fixation. Improved peripheral fixation can decrease central post micromotion and thereby enhance osseointegration.

Metal central posts or pegs offer an additional challenge if extraction is required, such as in cases of infection, revision, or conversion to reverse arthroplasty. Extraction of current metal designs can be difficult and can require removal of an undesirably large volume of bone. A trephine may be necessary to cut out a long cylinder, sacrificing additional bone and leaving a large diameter hole defect in the depths of the glenoid. In designs exemplified and presented herein, a cannulated, 3-tiered drill can be used to morsalize a porous metal coating of the Central Post using the solid core as a drill guide, thus minimizing bone loss. A cannulated drill can be similarly used to morsalize a Peripheral Peg porous coating using the solid central core as a drill guide.

Some prior, modular glenoid designs use uniformly sized central posts and uniformly sized peripheral pegs on implants of different sizes in patients with different sized glenoids. Oversizing posts and pegs in a small patient may lead to unwanted backside cortex perforation, which may compromise cement pressurization and/or risk of splitting the bony glenoid. Undersizing posts and pegs in a large patient may fail to fully utilize available bone and lead to non-optimal fixation, stability, osseointegration and durability Enhanced glenoid implant systems exemplified and presented herein can be used to select appropriate sized posts and pegs to implant with patient size to avoid these problems.

Modularity can be used to allow a surgeon to adjust for a condition seen at surgery, such as a better size-matching to patient anatomy. No prior designs use modularity in this fashion. Modularity, as it is currently used, permits attachment of either a one-sized fluted or one-sized metal central post to a common glenoid body, and can be undesirable for one or more of several reasons. Modularity requires time-consuming un-packaging and intraoperative assembly of an implant. A surgeon may have to stop progress with the case to perform the assembly or rely on a technician, who may not be familiar with device assembly. Operator error is a possibility Improper assembly or accidental dropping of or other damage to a small attachment with loss of sterility may occur. Quality control of the junctional attachment is typically performed on the operative back table, where manufacturing defects may either go unnoticed or be detected too late to rectify Implantation of a device with a junctional defect may cause premature device failure, recall, disability and result in possible litigation. Most prior enhanced ingrowth designs are non-modular and avoid modularity downsides. Prior designs, however, offer only one or the other enhanced ingrowth post options (flutes or metal). The one design that offers both flutes or metal options is modular, but has only one size post and carries the drawbacks previously mentioned. In the systems exemplified and presented herein, both porous metal and fluted designs may be options and can be non-modular. The components are assembled in the factory, where final quality control may be performed before distribution.

A cement-free glenoid implant option can be desirable and can eliminate time-consuming cement mixture, application and curing. A cementless device can avoid loosening problems associated with cement fatigue and facilitate extraction and revision. All previous attempts to acquire FDA approval for entirely cement-free, enhanced ingrowth implants have failed due to concerns with initial implant pull out strength and stability. All prior enhanced fixation glenoid implants require use of some cement around peripheral pegs or ridges. A reliable, highly stable, cement-free, enhanced ingrowth glenoid implant can be desired and is wanted in the marketplace, thus meeting a long-felt, unmet need in the field. More stable Central Post and Peripheral Pegs of systems presented herein can provide multiple points of fixation. Such features permit the cementless use of these glenoid implants.

Examples of enhanced glenoid implants and methods for their use are presented herein. Self-centering systems as described can increase implant stability and fixation, reduce micromotion, enhance osseointegration, improve functional result, extend long-term survival of the implant, and expand useful indications. In embodiments, non-modular, porous metal, and fluted options can utilize the same guides and central drills, and are designed to expedite implantation and extraction with minimal bone sacrifice. Size-matched, peg and post lengths and cemented or cementless options can provide a full spectrum of implant choices to optimally address individual patient needs and individual surgeon preference.

It can be readily appreciated that the descriptions and drawings herein are for purposes of illustration only and are not intended to limit the scope of these inventions. Rather, using the descriptions and teachings herein, other embodiments can be created by persons of skill in the art, and all such embodiments are considered part of this invention.

I claim:

1. A glenoid implant device, comprising:
a main body having a concave articular side and an opposite, convex side;
a central post affixed to said main body on said convex side, said central post having a cylindrical proximal portion having a first diameter, a middle cylindrical portion having a second diameter, and a cylindrical distal portion having a third diameter, said central post including:
a proximal portion having one or more coaxially arranged barbed disks thereon, at least one of said disks having a wider diameter proximally than distally on said central post;
a middle portion having one or more coaxially arranged flutes; and
a distal portion having one or more barbed disks thereon, at least one of said barbed disks having a wider diameter proximally than distally on said central post; and
one or more peripheral pegs, each of which is affixed to said main body on said convex side.

2. The device of claim 1, said first diameter being greater than said second diameter, and second diameter being greater than said third diameter.

3. The device of claim 1, at least one of said peripheral pegs having one or more barbed disks thereon having a wider diameter proximally than distally on said peripheral peg.

4. The device of claim 1, at least one of said glenoid implant, said central post, said stacked barbed disks, said peripheral pegs, and said flutes comprising polyethylene.

5. The device of claim 1, at least one of said flutes having one or more slits therein.

6. The device of claim 1 t where said glenoid implant is sized to fit the glenoid of a subject.

7. A glenoid implant device, comprising:
a main body having a concave articular side, and an opposite, convex side;
a central post affixed to said main body on said convex side, said central post having a cylindrical proximal portion having a first diameter, a middle cylindrical portion having a second diameter, and a cylindrical distal portion having a third diameter, said central post including:
a proximal portion having one or more coaxially arranged barbed disks thereon, at least one of said disks having a wider diameter proximally than distally on said central post;
a middle portion comprising a metal and having a diameter smaller than the diameter of said proximal portion of said central post;
a distal portion comprising a metal and having a diameter smaller than the diameter of said middle portion of said central post; and
one or more peripheral pegs, each of which is affixed to said main body on said convex side.

8. The device of claim 7, at least one of said central post or peripheral pegs comprising a metal.

9. The device of claim 7, at least one of said central post or peripheral pegs comprising a metal tip.

10. The device of claim 7, said metal being porous.

11. The device of claim 7, where said metal is titanium, stainless steel, cobalt chrome, tantalum, or other biocompatible metal.

12. The device of claim 7, said metal portion of said central post and at least one of said peripheral pegs being affixed to a proximal portion by way of a metal core.

13. The device of claim 12, said metal core having a screw attachment to said central post or said peripheral peg.

14. The device of claim 12, said metal core having a proximal helical blade attached to said central post or said peripheral peg.

15. The device of claim 12, said metal core having a proximal barb attached to said central post or said peripheral peg.

16. The device of claim 12, said metal core having a proximal textured surface attached to said central post or said peripheral peg.

17. The device of claim 1 being made as a single piece.

18. The device of claim 1 being modular.

19. A drill, comprising:
a handle;
a base having said handle attached thereto;
a 3-tiered cutting tip, having:
a proximal portion having a first diameter,
a middle portion having a second diameter; and
a distal portion having a third diameter;
said first diameter being greater than said second diameter; and
said second diameter being greater than said third diameter.

20. The drill of claim 19, said 3-tiered cutting tip further comprising at least one cutting groove.

21. A glenoid implant device, comprising:
a glenoid implant base;
means for affixing said base to a shoulder bone.
